(19) United States
(12) Patent Application Publication
(10) Pub. No.: US 2004/0186046 A1
(43) Pub. Date: Sep. 23, 2004

(54) Treatment of type 1 diabetes with PDE5 inhibitors

(75) Inventors: Gillian Munro Burgess, Sandwich (GB); Earl Michael Gibbs, Oakdale, CT (US); Christopher Peter Wayman, Sandwich (GB)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 10/799,191

(22) Filed: Mar. 12, 2004

Related U.S. Application Data
(60) Provisional application No. 60/455,277, filed on Mar. 17, 2003.

(30) Foreign Application Priority Data
Apr. 3, 2003 (GB) 0307740.1

(51) Int. Cl. A61K 38/28; A61K 31/505; A61K 31/401; A61K 31/366

(52) U.S. Cl. 514/3; 514/275; 514/408; 514/423; 514/460; 514/548

(57) ABSTRACT

The use of a PDE5 inhibitor without substantial PDE2 inhibiting activity, or a pharmaceutically acceptable salt thereof in the preparation of a medicament for the treatment of Type 1 Diabetes. A method of treating Type 1 Diabetes in an individual suffering from Type 1 Diabetes, which method comprises administering to said individual an effective amount of a PDE5 inhibitor without substantial PDE2 inhibiting activity, or a pharmaceutically acceptable salt thereof.

### CROSS-REFERENCE TO RELATED APPLICATION

This application claims priority from United Kingdom Application Number 0307740.1, filed on Apr. 3, 2003 and the benefit from U.S. Provisional Application No. 60/455,277, filed on Mar. 17, 2003.

### FIELD OF INVENTION

The invention described herein relates to the treatment of Type 1 diabetes, in particular to the treatment, including prophylaxis, palliative treatment and cure of Type 1 diabetes with an inhibitor of cGMP phosphodiesterase type 5 (also referred to as cGMP PDE5 or simply PDE5) without substantial PDE2 inhibiting activity.

A number of websites describe Type 1 diabetes, its etiology, treatments, etc., exemplified by:

http://www.diabetes.org;

http://www.genetichealth.com/DBTS What Is Type 1 Diabetes.shtml; and

http://www.merck.com/pubs/mmanual/section2/chapter13/13a.htm.

The information set forth therein is well known to those skilled in the art, is incorporated herein by reference, and paraphrased below.

Type 1 diabetes is usually diagnosed in children and young adults and is also known as juvenile diabetes, Type 1 diabetes mellitus (DM), insulin-dependent DM (IDDM) and juvenile-onset diabetes and may be known by other names. The term is used interchangeably herein. The peak time for developing diabetes is during puberty, although it can occur at any age. In Type 1 diabetes, the body does not produce insulin, or does not produce insulin in sufficient amounts. Insulin is necessary for the body to be able to use glucose, the basic fuel for the cells in the body, and insulin takes the glucose from the blood into the cells. The pancreas produces insulin in β-cells. Sometimes, the β-cells get destroyed or damaged and cannot produce (sufficient) insulin anymore. Many factors can cause damage to β-cells, but in most people with Type 1 diabetes, it is caused by a defect in the immune system. Cells that normally protect from germs attack the β-cells instead, which can die. Without β-cells, no insulin is made, sugar builds up in the blood, and diabetes develops. Patients with Type 1 diabetes may develop diabetic ketoacidosis (DKA).

Although researchers have found gene mutations that increase the risk of developing Type 1 diabetes, these genes alone do not cause the disease. There is probably therefore a combination of genetic risk and environmental factors (e.g. certain viruses) which cause the disease.

GENERAL CHARACTERISTICS OF THE MAJOR
CLINICAL TYPES OF DIABETES MELLITUS

| | Type 1 DM | |
|---|---|---|
| | (Insulin- | |
| | Dependent DM, | Type 2 DM |
| | Juvenile-Onset | (Non-Insulin- |
| Characteristic | Diabetes) | Dependent DM) |
| Age at onset | Most commonly | Most commonly |
| | <30 yr | >30 yr |
| Associated obesity | No | Very Common |
| Propensity to ketoacidosis | Yes | No |
| requiring insulin treatment | | |
| for its control | | |
| Endogenous insulin | Extremely low to | Significant but |
| secretion | undetectable plasma | variable levels of |
| | insulin and | insulin secretion |
| | C-peptide | that are low relative |
| | levels | to plasma glucose |
| | | levels and |
| | | accompanied by |
| | | insulin resistance |
| Twin concurrence | =50% | >90% |
| Associated with specific | Yes | No |
| HLA-D antigens | | |
| Islet cell antibodies at | Yes | No |
| diagnosis | | |
| Islet pathology | Insulitis, selective | Smaller, normal- |
| | loss of | appearing islets: |
| | most β cells | amyloid (amylin) |
| | | deposition is |
| | | common |
| Associated risks for | Yes | Yes |
| retinopathy, nephropathy, | | |
| neuropathy, and | | |
| atherosclerotic coronary and | | |
| peripheral vascular disease | | |
| in most Western populations | | |
| Hyperglycemia responds to | No | Yes, initially in |
| sulfonylureas | | many patients |

Common complications associated with or caused by diabetes include microvascular complications, retinopathy, nephropathy, peripheral and autonomic neuropathies, polyneuropathies, macrovascular complications including atherosclerotic coronary and peripheral arterial disease, foot ulcers and joint problems (e.g.Charcot's joints), increased risk of infection from fungi and bacteria (e.g. peripheral skin infections and oral and vaginal thrush) and renal failure.

Type 1 diabetics are typically dependent on insulin (usually by injection) to control their blood sugar levels.

### BACKGROUND

International Patent Application publications numbers WO02/22558, WO02/22559, WO02/022562, WO02/022561, and U.S. 2002019563 describe various nitric oxide synthase (NOS) inhibitors potentially useful in the treatment of type 1 diabetes and other conditions. JP 08208475 describes a nitrogen mono oxide inhibitor comprising 2-imino piperidine for treating type 1 diabetes, etc.

WO02/13798 discloses the use of PDE5 inhibitors in the treatment of type 2 diabetes, the insulin resistance syndrome, insulin resistance, and impaired glucose tolerance.

U.S. Pat. No. 6,479,493 discloses a method of treating Type 1 diabetes with a PDE2 inhibitor, optionally in the presence of a PDE5 inhibitor. Also disclosed therein is a use of a combined PDE2/PDE5 inhibitor in said treatment. The disclosure therein is said to be based on the presence of PDE2 and PDE5 in macrophages, the inhibition of which leads to apoptosis if said macrophages. In column 38 of U.S. Pat. No. 6,479,493 it is stated that “sildenafil (PDE5-specific Inhibitor) does not induce apoptosis. Therefore, the inhibition of only PDE5 alone (i.e. without the inhibition of PDE2) is not sufficient to induce apoptosis in U397 cells.”

International Patent Application publication number WO 00/23091 describes the use of agents which interfere with redistribution and/or targeting of inter alia, cGMP phosphodiesterases such as PDE5, but not their enzymic capacity, in the treatment of various conditions, such as Type 1 diabetes.

There remains a need for effective and straightforward treatment (including prophylaxis, palliative and cure) of Type 1 diabetes, and the conditions caused by Type 1 diabetes, such as those mentioned herein. There is also a need for a treatment to be administrable via alternative means to the injection methods commonly employed with respect to insulin.

### DESCRIPTION OF THE DRAWINGS

FIG. 1: Effect of sildenafil on average plasma glucose (mg/dl) in control and STZ -treated rats. Data was averaged from week 2 to week 6.

FIG. 2: Effect of sildenafil on minimum plasma glucose (mg/dl) in control and STZ-treated rats.

### DESCRIPTION OF THE INVENTION

We have unexpectedly discovered that use of a cGMP PDE5 inhibitor, without substantial PDE2 inhibiting activity, can be used in the treatment of Type 1 diabetes.

The invention is illustrated by the data below, which describes the effect of chronic delivery of sildenafil on blood sugar levels in rats pre-treated with streptozotocin (STZ). STZ is a nitrosamine which is toxic to the pancreatic β-cells and STZ-treated rats have been used as an experimental model for Type 1 diabetes (see e.g. Ari et al, Clin Sci (Colch) 1999:96:365).

### Study Design and Treatment Groups

| Treatment groups | | |
|---|---|---|
| Chronic treatment (sc) | CONTROL RATS | STZ RATS |
| Vehicle | G1 | G2 |
| Sildenafil SIL | G3 | G4 |
| (60 mg/kg/day sc) | | |

STZ: streptozotocin-induced Type 1 diabetes mellitus
G1 to G4: Number of experimental group

There were 12 rats in each of the control groups and 16 rats in the STZ groups.

### Induction of Diabetes

To induce Type 1 diabetes, rats of 200-250 g were injected intra-peritoneally with streptozotocin (60 mg/kg) in citrate phosphate buffer, pH4.5 or the equivalent volume of vehicle. The development of non-keturonic hyperglycaemic diabetes was induced within 48 h and was confirmed by determining blood glucose levels in blood obtained from the tail vein of the rats at 48 h after STZ administration and monitored bimonthly thereafter. STZ-treated rats were excluded from the study if their serum glucose levels were below below 15-20 nmol/L. Body weight was monitored weekly (diabetic rats fail to gain weight compared to control rats), but care was taken that their weight loss did not exceed 20%.

### Dosing

The dosing regimen consisted of sildenafil mesylate (<<SIL>>) or the equivalent volume of vehicle (<<VEH>>, physiological saline) given subcutanously, 20 mg/kg, three times a day targeted to give therapeutically meaningful plasma concentrations. The treatment with sildenafil commenced 48 h after administration of STZ and continued for a period of 8 weeks. The Sildenafil-treated rats were divided in two group—normal and STZ-treated. In addition, there were two non-sildenafil groups—again normal and STZ treated. Throughout this period blood samples were taken from the tail vein on a weekly basis. These samples were taken prior to dosing so that trough concentrations could be estimated (ie at 6-8 h post dose). A further sample was taken at week 8 (36-35 h post dosing). Plasma from these blood samples, from all rats treated with sildenafil, and some controls, were assayed for sildenafil (and its n-desmethyl metabolite). Results for sildenafil are shown below in Table 1.

TABLE 1

| | Sildenafil Mean plasma concentration | | | |
|---|---|---|---|---|
| | (nM; free drug) | | | |
| | | | Chronic sildenafil groups | |
| | Vehicle control | | (3 × 20 mg · kg sc/day) | |
| Week | groups | | Normal | STZ |
| (time post dose) | Normal | STZ | n = 12 | N = 6-10 |
| Baseline | — | <15 | <15 | <15 |
| 2 weeks (6-8 h) | — | — | 36 | 79 |
| 4 weeks (6-8 h) | — | — | 45 | 41 |
| 6 weeks (6-8 h) | — | — | 45 | 57 |
| 8 weeks (32-35 h) | — | <15 | 28 | 11 |

Analysis of plasma samples was performed at Pfizer and shows that clinically meaningful concentrations of sildenafil were achieved for the duration of the study. [The highest clinical dose of 100 mg corresponds to a plasma level of 40 nM). Results for both control and STZ-diabetic groups are broadly consistent, so there does not appear to be an effect of STZ on the pharmacokinetics of sildenafil. The free concentrations of the metabolite were similar to those of sildenafil ie circa 30-50 nM (data not shown).

### Measurement of Biological Endpoints

Plasma Glucose

Blood samples were taken into heparinised tubes before, weekly during the 8 week treatment period, in each case 6-8 h post dose and then at the end of the treatment, 32-35 h after the last dose of sildenafil or vehicle. The plasma from these samples was stored at −20° C. and analysed for levels of glucose using the Roche/Hitachi 912 Clinical Chemistry Analyzer (Roche Diagnostics Corp., Indianapolis, Ind.) with kits supplied by Roche. All assays were conducted according to instructions provided by the manufacturers.

### Results

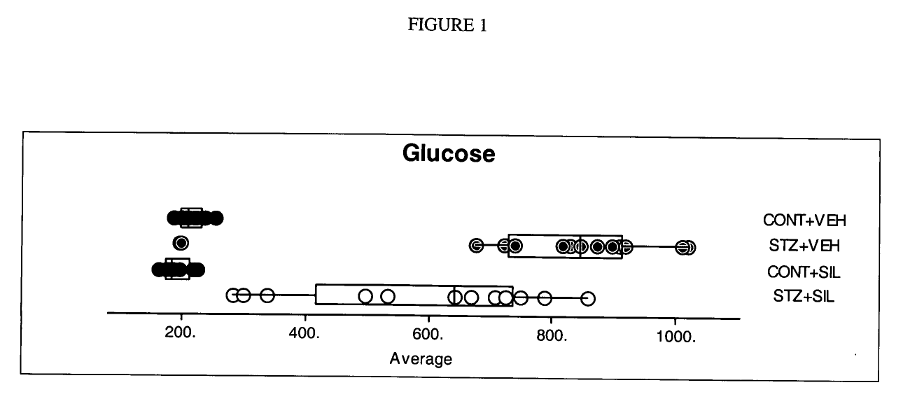

Effect of Sildenafil on plasma levels of glucose. FIG. 1 and Table 2 show the effect of sildenafil on average levels of plasma glucose in normal and STZ-treated rats.

(CONT=control; VEH=Vehicle); SIL=sildenafil)

There was a statistically significant interaction (p<0.05) between the type of rat (control or STZ-treated) and treatment received (vehicle or Sildenafil), meaning that the effect of Sildenafil was different in the two types of rat.

There was also a statistically significant effect of Sildenafil in both the control rats (p<0.01) and STZ-treated rats (p<0.05). Sildenafil lowered average glucose in both types of rat, but the overall decrease was greater in the STZ-treated animals than in the control animals.

TABLE 2
Effect of sildenafil on minimum plasma glucose
(mg/dl) in control and STZ-treated rats

| Type of | | | Change from | 95% Confidence |
|---|---|---|---|---|
| animal | Vehicle | Sildenafil | vehicle | Interval |
| Control | 215 | 190 | −25 | (−42, −7) |
| STZ-treated | 803 | 583 | −220 | (−382, −58) |

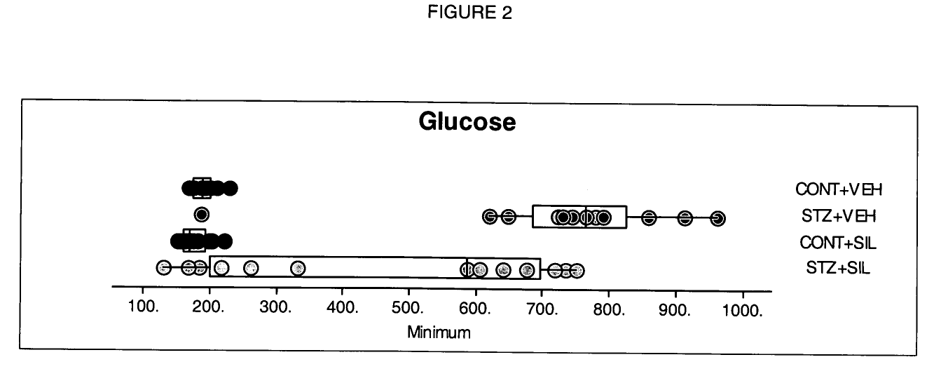

The effect of sildenafil on minimum glucose levels in the four groups of animals was also analysed (FIG. 2 and Table 3).

There was a statistically significant interaction (p<0.01) between the type of rat (control or STZ-treated) and treatment received (vehicle or Sildenafil), meaning that the effect of Sildenafil was different in the two types of rat.

There was no statistically significant effect of sildenafil on minimum glucose levels in the control rats (p>0.10) but a statistically significant effect in the STZ-treated animals (p<0.01).

TABLE 3

| | | | Change from | 95% Confidence |
|---|---|---|---|---|
| Type of animal | Vehicle | Sildenafil | vehicle | Interval |
| Control | 189 | 176 | −13 | (−30, 4) |
| STZ-treated | 731 | 462 | −269 | (−448, −91) |

This study supports the use of chronic sildenafil dosing in Type 1 diabetic mammals, including humans (men and women). The ability of sildenafil to lower glucose levels in this model of Type 1 diabetes suggest that sildenafil and other PDE5 inhibitors without substantial PDE2 inhibiting activity may have efficacy in this disease.

Aspects of the Invention Include:

1. The use of a PDE5 inhibitor without substantial PDE2 inhibiting activity, or a pharmaceutically acceptable salt thereof in the preparation of a medicament for the treatment of Type 1 diabetes;

2. A method of treating Type 1 diabetes in an individual suffering from Type 1 diabetes, which method comprises administering to said individual an effective amount of a PDE5 inhibitor without substantial PDE2 inhibiting activity, or a pharmaceutically acceptable salt thereof;

3. A pharmaceutical composition for use in the treatment of Type 1 diabetes comprising a PDE5 inhibitor without substantial PDE2 inhibiting activity, or a pharmaceutically acceptable salt thereof admixed with a pharmaceutically acceptable carrier, diluent or excipient;

4. A pharmaceutical combination (for simultaneous, separate or sequential administration) for the treatment of Type 1 diabetes in an individual comprising sildenafil or a pharmaceutically acceptable salt thereof and one or more additional agents active vs Type 1 diabetes or a condition caused by Type 1 diabetes; and

5. A kit for the treatment of Type 1 diabetes comprising a PDE5 inhibitor without substantial PDE2 inhibiting activity, or a pharmaceutically acceptable salt thereof, in an effective amount, optionally one or more pharmaceutically acceptable carrier, excipient or diluent, and one or more of:

a. a means for testing for Type 1 diabetes;

b. one or more additional agents agents active vs Type 1 diabetes or a condition caused by Type 1 diabetes; and/or

c. instructions for the treatment of Type 1 diabetes.

By “a PDE5 inhibitor without substantial PDE2 inhibiting activity” we mean compounds with PDE5 inhibition IC₅₀'s of less than 100 nanomolar, more preferably, at less than 50 nanomolar, more preferably still at less than 10 nanomolar, and that the compound has a selectivity for inhibiting PDE5 vs PDE2 of at least 30× as measured by comparing the IC₅₀ values vs these enzymes, i.e. the IC₅₀ value vs PDE5 for the compound is at least 30 times smaller than the corresponding IC₅₀ value vs PDE2. Preferably the PDE5/PDE2 selectivity is at least 100×, more preferably at least 1000×.

The suitability of any particular cGMP PDE5 inhibitor without substantial PDE2 inhibiting activity can be readily determined by evaluation of its potency and selectivity using the methods described herein, literature methods, etc., followed by evaluation of its toxicity, absorption, metabolism, pharmacokinetics, etc in accordance with standard pharmaceutical practice.

Preferably the cGMP PDE5 inhibitors without substantial PDE2 inhibiting activity used in the pharmaceutical combinations according to the present invention are also selective vs other PDEs. Preferably they have a selectivity of PDE5 over PDE3 of greater than 100 more preferably greater than 300. More preferably the PDE5 has a selectivity over both PDE3 and PDE4 of greater than 100, more preferably greater than 300.

Selectivity ratios may readily be determined by the skilled person. For example, IC50 values for the PDE3 and PDE4 enzyme may be determined using established literature methodology, see S A Ballard et al, Journal of Urology, 1998, vol.159, pages 2164-2171 and as detailed herein after.

Measurement of PDE5, PDE2, etc. inhibition is illustrated by the following assays.

### Assays

Compounds suitable for use in accordance with the present invention are potent and selective PDE5 inhibitors without substantial PDE2 inhibiting activity. In vitro PDE inhibitory activities against cyclic guanosine 3′,5′-monophosphate (CGMP) and cyclic adenosine 3′,5′-monophosphate (cAMP) phosphodiesterases can be determined by measurement of their IC₅₀ values (the concentration of compound required for 50% inhibition of enzyme activity).

The required PDE enzymes can be isolated from a variety of sources, including human corpus cavernosum, human and rabbit platelets, human cardiac ventricle, human skeletal muscle and bovine retina, essentially by the method of W. J. Thompson and M. M. Appleman (Biochem., 1971, 10, 311). In particular, the cGMP-specific PDE (PDE5) and the cGMP-inhibited cAMP PDE (PDE3) can be obtained from human corpus cavernosum tissue, human platelets or rabbit platelets; the cGMP-stimulated PDE (PDE2) can be obtained from human corpus cavernosum, the calcium/calmodulin (Ca/CAM)-dependent PDE (PDE1) from human cardiac ventricle; the cAMP-specific PDE (PDE4) from human. skeletal muscle; and the photoreceptor PDE (PDE6) from bovine retina. Phosphodiesterases. 7-11 can be generated from full length human recombinant clones transfected into SF9 cells.

Assays can be performed either using a modification of the “batch” method of W. J. Thompson et al. (Biochem., 1979, 18, 5228) or using a scintillation proximity assay for the direct detection of AMP/GMP using a modification of the protocol described by Amersham plc under product code TRKQ7090/7100. In summary, the effect of PDE inhibitors can be investigated by assaying a fixed amount of enzyme in the presence of varying inhibitor concentrations and low substrate, (cGMP or cAMP) in a 3:1 ratio unlabelled to [³H]-labeled at a conc ˜⅓ Kₘ) such that IC₅₀≅Kᵢ. The final assay volume is made up to 100 μl with assay buffer [20 mM Tris-HCl pH 7.4, 5 mM MgCl₂, 1 mg/ml bovine serum albumin]. Reactions are initiated with enzyme, incubated for 30-60 min at 30° C. to give <30% substrate turnover and terminated with 50 μl yttrium silicate SPA beads (containing 3 mM of the respective unlabelled cyclic nucleotide for PDEs 9 and 11). Plates are re-sealed and shaken for 20 min, after which the beads are allowed to settle for 30 min in the dark and then counted on a TopCount plate reader (Packard, Meriden, Conn.). Radioactivity units are converted to % activity of an uninhibited control (100%), plotted against inhibitor concentration and inhibitor IC₅₀ values obtained using the ‘Fit Curve’ Microsoft Excel extension.

Functional Activity

This can be assessed in vitro by determining the capacity of a PDE5 inhibitor of the invention to enhance sodium nitroprusside or electric field stimulation-induced relaxation of pre-contracted rabbit corpus cavernosum tissue strips, as described by S. A. Ballard et al. (Brit. J. Pharmacol., 1996, 118 (suppl.), abstract 153P) or S. A. Ballard et al. (J. Urology, 1998, vol. 159, 2164-2171).

### In Vitro PDE Inhibitory Activities

In vitro PDE inhibitory activities against cyclic guanosine 3′,5′-monophosphate (cGMP) phosphodiesterases can be determined by measurement of their IC₅₀ values (the concentration of compound required for 50% inhibition of enzyme activity).

As used herein, the terms “pharmaceutical” and “pharmaceutically” may include “veterinary” and “veterinarily”, respectively.

It is to be understood that all references herein to treatment include one or more of curative, palliative and prophylactic treatment. Preferably, the term treatment includes at least curative treatment and/or palliative treatment.

Further, it is to be appreciated that all references herein to treatment include acute treatment (taken as required) and chronic treatment (longer term continuous treatment).

Preferably the treatment provides a chronic level of PDE5 inhibition without substantial PDE2 inhibition in the treatment of Type 1 diabetes.

Chronic levels of PDE5 inhibition may be provided by daily multidosing of PDE5 inhibitors, by use of a PDE5 inhibitor which has a long half-life, by use of a formulation or device which provides for sustained or controlled or pulsatile release of the PDE5 inhibitor, or other means well-known in the art.

Suitable PDE5 inhibitors without substantial PDE2 inhibiting activity for the use according to the present invention may be any that satisfy the definition given above, and may include:

the pyrazolo [4,3-d]pyrimidin-7-ones disclosed in EP-A-0463756; the pyrazolo [4,3-d]pyrimidin-7-ones disclosed in EP-A-0526004; the pyrazolo [4,3-d]pyrimidin-7-ones disclosed in published international patent application WO 93/06104; the isomeric pyrazolo [3,4-d]pyrimidin-4-ones disclosed in published international patent application WO 93/07149; the quinazolin-4-ones disclosed in published international patent application WO 93/12095; the pyrido [3,2-d]pyrimidin-4-ones disclosed in published international patent application WO 94/05661; the purin-6-ones disclosed in published international patent application WO 94/00453; the pyrazolo [4,3-d]pyrimidin-7-ones disclosed in published international patent application WO 98/49166; the pyrazolo [4,3-d]pyrimidin-7-ones disclosed in published international patent application WO 99/54333; the pyrazolo [4,3-d]pyrimidin-4-ones disclosed in EP-A-0995751; the pyrazolo [4,3-d]pyrimidin-7-ones disclosed in published international patent application WO 00/24745; the pyrazolo [4,3-d]pyrimidin-4-ones disclosed in EP-A-0995750; the hexahydropyrazino [2′,1′:6,1]pyrido [3,4-b]indole-1,4-diones disclosed in published international application WO95/19978; the pyrazolo [4,3-d]pyrimidin-4-ones disclosed in WO00/27848; the imidazo[5,1-f][1,2,4]triazin-ones disclosed in EP-A-1092719 and in published international application WO 99/24433 and the bicyclic compounds disclosed in published international application WO 93/07124; the pyrazolo [4,3-d]pyrimidin-7-ones disclosed in published international application WO 01/27112; the pyrazolo [4,3-d]pyrimidin-7-ones disclosed in published international application WO 01/27113; the compounds disclosed in EP-A-1092718 and the compounds disclosed in EP-A-1092719; the tricyclic compounds disclosed in EP-A-1241170; the alkyl sulphone compounds disclosed in published international application WO 02/074774; the compounds disclosed in published international application WO 02/072586; the compounds disclosed in published international application WO 02/079203 and the compounds disclosed in WO 02/074312.

Preferred type V phosphodiesterase inhibitors (PDE5 inhibitors) for the use according to the present invention include:

5-[2-ethoxy-5-(4-methyl-1-piperazinylsulphonyl)phenyl]-1-methyl-3-n-propyl-1,6-dihydro-7H-pyrazolo[4,3-d]pyrimidin-7-one (sildenafil, e.g. as sold as Viagra®) also known as 1-[[3-(6,7-dihydro-1-methyl-7-oxo-3-propyl-1H-pyrazolo[4,3-d]pyrimidin-5-yl)-4-ethoxyphenyl]sulphonyl]-4-methylpiperazine (see EP-A-0463756);

5-(2-ethoxy-5-morpholinoacetylphenyl)-1-methyl-3-n-propyl-1,6-dihydro-7H-pyrazolo[4,3-d]pyrimidin-7-one (see EP-A-0526004);

3-ethyl-5-[5-(4-ethylpiperazin-1-ylsulphonyl)-2-n-propoxyphenyl]-2-(pyridin-2-yl)methyl-2,6-dihydro-7H-pyrazolo[4,3-d]pyrimidin-7-one (see WO98/49166);

3-ethyl-5-[5-(4-ethylpiperazin-1-ylsulphonyl)-2-(2-methoxyethoxy)pyridin-3-yl]-2-(pyridin-2-yl)methyl-2,6-dihydro-7H-pyrazolo[4,3-d]pyrimidin-7-one (see WO99/54333);

(+)-3-ethyl-5-[5-(4-ethylpiperazin-1-ylsulphonyl)-2-(2-methoxy-1(R)-methylethoxy)pyridin-3-yl]-2-methyl-2,6-dihydro-7H-pyrazolo[4,3-d]pyrimidin-7-one, also known as 3-ethyl-5-{5-[4-ethylpiperazin-1-ylsulphonyl]-2-([(1R)-2-methoxy-1-methylethyl]oxy)pyridin-3-yl}-2-methyl-2,6-dihydro-7H-pyrazolo[4,3-d]pyrimidin-7-one (see WO99/54333);

5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one, also known as 1-{6-ethoxy-5-[3-ethyl-6,7-dihydro-2-(2-methoxyethyl)-7-oxo-2H-pyrazolo[4,3-d]pyrimidin-5-yl]-3-pyridylsulphonyl}-4-ethylpiperazine (see WO 01/27113, Example 8);

5-[2-iso-Butoxy-5-(4-ethylpiperazin-1-ylsulphonyl)pyridin-3-yl]-3-ethyl-2-(1-methylpiperidin-4-yl)-2,6-dihydro-7H-pyrazolo[4,3-d]pyrimidin-7-one (see WO 01/27113, Example 15);

5-[2-Ethoxy-5-(4-ethylpiperazin-1-ylsulphonyl)pyridin-3-yl]-3-ethyl-2-phenyl-2,6-dihydro-7H-pyrazolo[4,3-d]pyrimidin-7-one (see WO 01/27113, Example 66);

5-(5-Acetyl-2-propoxy-3-pyridinyl)-3-ethyl-2-(1-isopropyl-3-azetidinyl)-2,6-dihydro-7H-pyrazolo[4,3-d]pyrimidin-7-one (see WO 01/27112, Example 124);

5-(5-Acetyl-2-butoxy-3-pyridinyl)-3-ethyl-2-(1-ethyl-3-azetidinyl)-2,6-dihydro-7H-pyrazolo[4,3-d]pyrimidin-7-one (see WO 01/27112, Example 132);

(6R, 12aR)-2,3,6,7,12,12a-hexahydro-2-methyl-6-(3,4-methylenedioxyphenyl) pyrazino[2′,1′:6,1]pyrido[3,4-b]indole-1,4-dione (tadalafil, IC-351, Cialis®), i.e. the compound of examples 78 and 95 of published international application WO95/19978, as well as the compound of examples 1, 3, 7 and 8;

2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil, LEVITRA®) also known as 1-[[3-(3,4-dihydro-5-methyl-4-oxo-7-propylimidazo[5,1-f]-as-triazin-2-yl)-4-ethoxyphenyl]sulphonyl]-4-ethylpiperazine, i.e. the compound of examples 20, 19, 337 and 336 of published international application WO99/24433;

the compound of example 11 of published international application WO93/07124 (EISAI);

compounds 3 and 14 from Rotella D P, J. Med. Chem., 2000, 43, 1257;

4-(4-chlorobenzyl)amino-6,7,8-trimethoxyquinazoline;

N-[[3-(4,7-dihydro-1-methyl-7-oxo-3-propyl-1H-pyrazolo[4,3-d]-pyrimidin-5-yl)-4-propxyphenyl]sulfonyl]-1-methyl2-pyrrolidinepropanamide [“DA-8159” (Example 68 of WO00/27848)]; and

7,8-dihydro-8-oxo-6-[2-propoxyphenyl]-1H-imidazo[4,5-g]quinazoline and 1-[3-[1-[(4-fluorophenyl)methyl]-7,8-dihydro-8-oxo-1H-imidazo[4,5-g]quinazolin-6-yl]-4-propoxyphenyl]carboxamide.

Still other type, cGMP PDE5 inhibitors which may be useful in conjunction with the present invention include:4-bromo-5-(pyridylmethylamino)-6-[3-(4-chlorophenyl)-propoxy]-3(2H )pyridazinone; 1-[4-[(1,3-benzodioxol-5-ylmethyl)amiono]-6-chloro-2-quinozolinyl]-4-piperidine-carboxylic acid, monosodium salt; (+)-cis-5,6a,7,9,9,9a-hexahydro-2-[4-(trifluoromethyl)-phenylmethyl-5-methyl-cyclopent-4,5]imidazo[2,1-b]purin-4(3H )one; furazlocillin; cis-2-hexyl-5-methyl-3,4,5,6a,7,8,9,9a-octahydrocyclopent[4,5]-imidazo[2,1-b]purin-4-one; 3-acetyl-1-(2-chlorobenzyl)-2-propylindole-6-carboxylate; 3-acetyl-1-(2-chlorobenzyl)-2-propylindole-6-carboxylate; 4-bromo-5-(3-pyridylmethylamino)-6-(3-(4-chlorophenyl) propoxy)-3-(2H)pyridazinone; 1-methyl-5(5-morpholinoacetyl-2-n-propoxyphenyl)-3-n-propyl-1,6-dihydro-7H-pyrazolo(4,3-d)pyrimidin-7-one; 1-[4-[(1,3-benzodioxol-5-ylmethyl)amino]-6-chloro-2-quinazolinyl]-4-piperidinecarboxylic acid, monosodium salt; Pharmaprojects No. 4516 (Glaxo Wellcome); Pharmaprojects No. 5051 (Bayer); Pharmaprojects No. 5064 (Kyowa Hakko; see WO 96/26940); Pharmaprojects No. 5069 (Schering Plough); GF-196960 (Glaxo Wellcome); E-8010 and E-4010 (Eisal); Bay-38-3045 & 38-9456 (Bayer); FR229934 and FR226807 (Fujisawa); and Sch-51866.

More preferably the PDE5 inhibitor without substantial PDE2 inhibiting activity is selected from sildenafil, tadalafil, vardenafil, DA-8159 and 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one.

Most preferably the PDE5 inhibitor without substantial PDE2 inhibiting activity is sildenafil.

It is to be understood that the contents of the above published patent applications, and in particular the general formulae and exemplified compounds therein are incorporated herein in their entirety by reference thereto.

### Combinations

The pharmaceutical compositions for use in the invention may additionally comprise one or more additional active agents. The present invention further comprises the use of combinations of the PDE5 inhibitor without substantial PDE2 inhibiting activity for the treatment of Type 1 diabetes with one or more additional active agents (for simultaneous, separate or sequential administration) effective in the treatment of Type 1 diabetes and/or its downstream consequences/conditions.

Thus, references herein to the use of PDE5 inhibitors without substantial PDE2 inhibiting activity for use according to the present invention also includes combination of PDE5 inhibitors without substantial PDE2 inhibiting activity with other additional (active) agents.

Such additional agent may be another Type 1 diabetes drug as detailed herein, such as for example clomid.

Combinations of PDE5 inhibitors without substantial PDE2 inhibiting activity, useful for the treatment of Type 1 diabetes according to the present invention, with an additional agent are discussed in more detail below.

The method of the present invention may also be used in conjunction with hormone therapy. By way of example, the present invention may be used in conjunction with one or more hormones or steroids—such as those mentioned in WO-A-99/21562.

Additional active agents which may be suitable for use in the present invention include the following:

(i) one or more NO-donor (NO-agonist) compounds or NO synthase substrate, such as organic nitrates, such as mono- di or tri-nitrates or organic nitrate esters including glyceryl trinitrate (also known as nitroglycerin), isosorbide 5-mononitrate, isosorbide dinitrate, pentaerythritol, tetranitrate, erythrityl tetranitrate, sodium nitroprusside (SNP), 3-morpholinosydnonimine molsidomine, S-nitroso- N-acetyl penicilliamine (SNAP) S-nitroso-N-glutathione (SNO-GLU), N-hydroxy-L-arginine, amylnitrate, linsidomine, linsidomine chlorohydrate, (SIN-1) S-nitroso-N-cysteine, diazenium diolates,(NONOates), 1,5-pentanedinitrate, L-arginine, folic acid, ginseng, zizphi fructus, molsidomine, Re-2047, nitrosylated maxisylyte derivatives such as NMI-678-11 and NMI-937 as described in published PCT application WO 0012075; and/or

(ii) one or more potassium channel openers or modulators. Suitable potassium channel openers/modulators for use herein include nicorandil, cromokalim, levcromakalim, lemakalim, pinacidil, cliazoxide, minoxidil, charybdotoxin, glyburide, 4-amini pyridine, BaCl₂; and/or

(iii) one or more angiotensin receptor antagonists such as losartan; and/or

(iv) one or more antilipemic agents (see below); and/or

(v) one or more antiplatelet and antithrombotic agents, e.g. tPA, uPA, warfarin, hirudin and other thrombin inhibitors, heparin, thromboplastin activating factor inhibitors; and/or

(vi) one or more insulin sensitising agents such as Rezulin, Avandia or Actos and hypoglycaemic agents such as, but not limited to, glipizide (sulfonylureas), metformin, or acarbose; and/or

(vii) one or more acetylcholinesterase inhibitors such as donezipil; and/or

(viii) one or more estrogen receptor modulators and/or estrogen agonists and/or estrogen antagonists, preferably raloxifene or lasofoxifene, (−)-cis-6-phenyl-5-[4-(2-pyrrolidin-1-yl-ethoxy)-phenyl]-5,6,7,8-tetrahydronaphthalene-2-ol and pharmaceutically acceptable salts thereof (compound A below) the preparation of which is detailed in WO 96/21656;

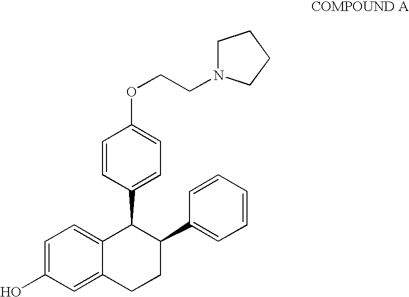

Compound A

(ix) one or more of a further PDE inhibitor selected from a PDE 4, 7 or 8 inhibitor, said inhibitors preferably having an IC50 against the respective enzyme of less than 100 nM: and/or

(x) one or more of an NEP inhibitor, preferably wherein said NEP is EC 3.4.24.11 and more preferably wherein said NEP inhibitor is a selective inhibitor for EC 3.4.24.11, which has an IC₅₀ vs NEP of less than 100 nM (e.g. ompatrilat, sampatrilat)—suitable NEP inhibitor compounds are described in EP-A-1097719; and/or

(xi) one or more compounds which inhibit angiotensin-converting enzyme such as enalapril, and one or more combined inhibitors of angiotensin-converting enzyme and neutral endopeptidase such as omapatrilat; and/or

(xii) one or more calcium-channel blockers such as amlodipine; and/or

(xiii) pregabalin; gabapentin; and/or

(xiv) one or more protein kinase C-β inhibitors such as LY333531; and/or

(xv) one or more activators of AMP-activated protein kinase such as 5-amino-4-imidazolecarboxamide ribonucleoside; and/or

(xvi) insulin (suitable for inhalation or injection, etc.); and/or

(xvii) weight loss agents such as sibutramine or orlistat; and/or

(xviii) one or more dipeptidyl peptidase IV inhibitors such as NVP DPP728 or P32/98; and/or

(xix) one or more glucagon antagonists such as NNC25-2504; and/or

(xx) one or more agents that inhibit PTP1B such as PTP112; and/or

(xxi) one or more agents that reduce PTP1B levels using antisense technology; and/or

(xxii) one or more glycogen synthase kinase-3 inhibitors such as Chir98014; and/or

(xxiii) one or more GLP-1 agonists such as GLP1, NN-2211 or exendin 4; and/or

(xxiv) one or more PPAR-gamma agonists such as Rezulin, Avandia, Actos or CS011; and/or

(xxv) one or more PPAR-alpha agonists such as fenofibrate; and/or

(xxvi) one or more dual PPAR-alpha/PPAR-gamma agonists such as farglitazar, rosiglitasone, pioglitazone, darglitazone, GW1929, DRF2725, AZ242 or KRP 297; and/or

(xxvii) one or more sorbitol dehydrogenase inhibitors such as 1R-(4-4,6-dimethy-[1,3,5}triaziny-2-yl)-2R,6S-dimethylpiperazin-1-yl)pyrimidin-2-yl)-ethanol (see e.g. WO/0059510); and/or

(xxviii) one or more aldose reductase inhibitors such as 6-(5-chloro-3-methylbenzofuran-2-sulphonyl)-2H-pyridazin-3-,one (see e.g. WO02/079198), zopolrestat, zenarestat, or fidarestat; and/or

(xxix) soluble guanyl cyclase (sGC) activators such as BAY 41-8543 and BAY 41-2272.

Combinations of PDE5 inhibitors without substantial PDE2 inhibiting activity with agents mentioned in sections (iv), (viii) and (xvi) above are preferred.

### Antilipemic Agents

According to the present invention, the antilipemic agents mentioned in (iv) above can be selected from the group consisting of:

HMG-CoA-reductase inhibitors

squalene synthase inhibitors,

bile acid absorption inhibitors (also referred to as “bile acid anion exchangers” or bile acid sequestrants),

fibric acid and its derivatives,

nicotinic acids and its analogs and also

ω3-fatty acids.

For further details about the antilipemics mentioned above, reference is in this context made to the article by Gilbert R. Thompson and Rissitaza P. Naoumova “New prospects for lipid-lowering drugs” in Exp. Opin. Invest. Drugs (1998), 7 (5), pages 715-727, the entire content of which is hereby expressly incorporated by way of reference. Among the antilipemics mentioned above, preference according to the invention is given to the HMG-CoA-reductase inhibitors. Here, the abbreviation “HMG-CoA” denotes “3-hydroxymethylglutaryl-coenzyme A”.

Among the HMG-CoA-reductase inhibitors, in turn, preference according to the invention is given, in particular, to the substance class of the vastatins—which, for the sake of simplicity, are in most cases referred to in the literature simply as “statins”.

Among the statins, in turn, particular preference according to the invention is given to,

atorvastatin (commercially available under the name Lipitor® from Parke-Davis/Pfizer);

cerivastatin (commercially available under the name Lipobay® or Baycol from Bayer);

fluvastatin (commercially available under the name Lescol® from Novartis);

lovastatin (commercially available under the name Mevacor® from Merck);

pravastatin (commercially available under the name Lipostat® from Bristol-Myers squibb);

simvastatin (commercially available under the name Zocor® from Merck);

itavastatin (also called “nisvastatin”; NK-104; systematic name: [S-[R*,S*-(E)]]-7-[2-cyclopropyl-4-(4-fluorophenyl)-3-quinolinyl]-3,5-dihydroxy-6-heptenoic acid);

dalvastatin;

mevastatin;

dihydrocompactin;

compactin; and

(+)-(3R, 5S)-bis-(7-(4-(4-fluorophenyl)-6-isopropyl-2-(N-methyl-N-methanesulfonylamino)-pyrimidin-5-yl)-3,5-dihydroxy-6(E)-heptenoic acid; and their respective salts, hydrates, alkoxides, esters and tautomers, and among these with very particular preference atorvastatin, cerivastatin, fluvastatin, lovastatin, pravastatin, itavastatin, simvastatin and (+)-(3R,5S)-bis-(7-(4-(4-fluorophenyl)-6-isopropyl-2-(N-methyl-N-methanesulfonylamino)-pyrimidin-5-yl)-3,5-dihydroxy-6(E)-heptenoic acid and their respective salts, hydrates, alkoxides, esters and tautomers.

Among these, in turn, very particular preference is given to cerivastatin and atorvastatin and their respective salts, hydrates, alkoxides, esters and tautomers. For further details about the statins mentioned above, reference is made to the discourse in Drugs of the Future 1994, 19(6), pages 537-541 and 1995, 20(6), page 611 and 1996, 21(6), page 642, the respective content of which is included herein in its entirety by way of reference.

For the purpose of the present invention, with regard to antilipemic agents, the term “salt” refers in each case to physiologically acceptable salts of the compounds in question: these can, for example, be salts with mineral acids, carboxylic acids or sulfonic acids, in particular with hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, propionic acid, lactic acid, tartaric acid, citric acid, fumaric acid, maleic acid or benzoic acid, or else mixed salts thereof. However, the salts can also be salts with customary bases, such as, for example, alkali metal salts (for example sodium or potassium salts), alkaline earth metal salts (for, example calcium or magnesium salts) or ammonium salts, derived from ammonia or organic amines, such as, for example, diethylamine, triethylamine, ethyldiisopropylamine, procaine, dibenzylamine, N-methylmprpholine, dihydroabietylamine, 1-ephenamine or methyl-piperidine, and also mixed salts thereof.

Examples of statin salts which can be used according to the invention are fluindostatin (the monosodium salt of fluvastatin); the monopotassium salt and the calcium salt of itavastatin; and the calcium salt of (+)-(3R,5S)-bis-(7-(4-(4-fluorophenyl)-6-isopropyl-2-(N-methyl-N-methane-sulfonylamino)-pyrimidin-5-yl)-3,5-dihydroxy-6(E)-heptenoic acid (“ZD 4522” or “S 4522” from Shionogi and AstraZeneca, respectively). Further examples of statin salts which can be used according to the invention are the monosodium and the monopotassium salts and also the calcium salts of cerivastatin, atorvastatin and pravastatin.

Further preferred HMG-CoA-reductase inhibitors are described in EP-A-0 325 130 and EP-A-0-491 226, both in the name of Bayer AG, the content of which is hereby included by way of reference. EP-A-0 325 130 provides substituted pyridines, and EP-A-0-491 226 describes substituted pyridyldihydroxyheptenoic acid derivatives and their salts, and among these in particular cerivastatin, which is particularly preferred according to the invention (claim 6 of EP-A-0-491 226).

Preference according to the invention is also given to the statins mentioned in WO-A-99/11263, the disclosure of which is included by way of reference.

Preference according to the invention is likewise given to the HMG-CoA-reductase inhibitors mentioned in the publication Bioorganic & Medicinal Chemistry, Vol. 5, No. 2, pages 437-444 (1997), the disclosure of which is hereby included in its entirety by way of reference.

A further review of HMG-CoA-reductase inhibitors can be found in Pharmazie in unserer Zeit, Vol.28, No. 3, pages 147-1152 (1999).

Among the bile acid sequestrants mentioned above, preference according to the invention is given to cholestyramine (commercially available under the name Questran® from Bristol-Myers Squibb) and colestipol (commercially available under the name Colestid® from Pharmacia & Upjohn) (see also Exp. Opin, Invest. Drugs (1998), 7(5) pages 715-727).

Among the fibric acid derivatives mentioned above, preference according to the invention is given to ciprofibrate (commercially available under the name Modalim® from Sanofi Winthorp), fenofibrate (commercially available under the name Lipantil® from. Fournier), gemfibrozil (commercially available under the name Lopid® from Parke-Davis), bezafibrate and chlofibrate (see also Exp. Opin. Invest. Drugs (1998), 7(5), pages 715-727.

Among the nicotinic acid analogs mentioned above, preference according to the invention is given to acipimox (commercially available under the name Olbetam® from Pharmacia & Upjohn) (see also Exp. Opin. Invest. Drugs (1998), 7(5), pages 715-727).

Among the ω3-fatty acids mentioned above, preference according to the invention is given to Maxepa (distributed by Seven Seas) (see also Exp. Opin. Invest. Drugs (1998), 7(5), pages 715-727).

Preferred specific combinations for use in accordance with the invention comprise:

(i) any PDE5 inhibitor without substantial PDE2 inhibiting activity selected from sildenafil, tadalafil, vardenafil, DA-8159 and 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one and their respective salts, solvates,

(ii) with any one of: insulin, raloxifene, lasofoxifene, (−)-cis-6-phenyl-5-[4-(2-pyrrolidin-1-yl-ethoxy)-phenyl]-5,6,7,8-tetrahydronaphthalene-2-ol, atorvastatin, cerivastatin, fluvastatin, lovastatin, pravastatin, itavastatin, simvastatin and (+)-(3R,5S)-bis-(7-(4-(4-fluorophenyl)-6-isopropyl-2-(N-methyl-N-methanesulfonylamino)-pyrimidin-5-yl)-3,5-dihydroxy-6(E)-heptenoic acid and their respective salts, hydrates, alkoxides, esters and tautomers.

The cGMP PDE5 inhibitor without substantial PDE2 inhibiting activity, either alone or in combination with one or more additional active agent, such as those mentioned above, are referred to as “agents of the invention”. If a combination of active agents is administered, they can be delivered to the treatment subject in the same formulation and in the same administration method, or they can be delivered in separate formulations and by the same or different methods.

Pharmaceutically acceptable salts of the agents of the invention include the acid addition and base salts (including disalts) thereof. Suitable acid addition salts are formed from acids which form non-toxic salts. Examples include the acetate, aspartate, benzoate, besylate, bicarbonate/carbonate, bisulphate, camsylate, citrate, edisylate, esylate, fumarate, gluceptate, gluconate, glucuronate, hibenzate, hydrochloride/chloride, hydrobromide/bromide, hydroiodide/iodide, hydrogen phosphate, isethionate, D- and L-lactate, malate, maleate, malonate, mesylate, methylsulphate, 2-napsylate, nicotinate, nitrate, orotate, palmoate, phosphate, saccharate, stearate, succinate sulphate, D- and L-tartrate, and tosylate salts. Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. For a review on suitable salts, see Stahl and Wermuth, Handbook of Pharmaceutical Salts: Properties,.Selection, and Use, Wiley-VCH, Weinheim, Germany (2002). A pharmaceutically acceptable salt of an agent of the invention may be readily prepared by mixing together solutions of the agent and the desired acid or base, as appropriate. The salt may precipitate from solution and be collected by filtration or may be recovered by evaporation of the solvent.

Pharmaceutically acceptable solvates for use in accordance with the invention include hydrates and solvates wherein the solvent of crystallization may be isotopically substituted, e.g. D₂O, d₆-acetone, d₆-DMSO.

Also within the scope of the invention is the use of clathrates, drug-host inclusion complexes wherein, in contrast to the aforementioned solvates, the drug and host are present in non-stoichiometric amounts. For a review of such complexes, see J Pharm Sci, 64 (8),1269-1288 by Haleblian (August 1975).

Herein all references to agents of the invention include references to salts thereof and to solvates and clathrates of agents of the invention and salts thereof.

The invention includes the use of all polymorphs of the agents of the invention as hereinbefore defined.

Also within the scope of the invention is the use of so-called “prodrugs” of the agents of the invention. Thus certain derivatives of agents of the invention which have little or no pharmacological activity themselves can, when metabolised upon administration into or onto the body, give rise to agents of the invention having the desired activity. Such derivatives are referred to as “prodrugs”.

Prodrugs for use in accordance with the invention can, for example, be produced by replacing appropriate functionalities present in the agents of the invention with certain moieties known to those skilled in the art as “pro-moieties” as described, for example, in “Design of Prodrugs” by H Bundgaard (Elsevier, 1985).

Certain agents of the invention may themselves act as prodrugs of other agents of the invention.

Agents of the invention containing one or more asymmetric carbon atoms can exist as two or more optical isomers. Where an agent of the invention contains an alkenyl or alkenylene group, geometric cis/trans (or Z/E) isomers are possible, and where the compound contains, for example, a keto or oxime group, tautomeric isomerism (‘tautomerism’) may occur. It follows that a single compound may exhibit more than one type of isomerism.

Included within the scope of the present invention are the use of all optical isomers, geometric isomers and tautomeric forms of the agents of the invention, including compounds exhibiting more than one type of isomerism, and mixtures of one or more thereof.

Cis/trans isomers may be separated by conventional techniques well known to those skilled in the art, for example, fractional crystallisation and chromatography.

Conventional techniques for the preparation/isolation of individual stereoisomers include the conversion of a suitable optically pure precursor, resolution of the racemate (or the racemate of a salt or derivative) using, for example, chiral HPLC, or fractional crystallisation of diastereoisomeric salts formed by reaction of the racemate with a suitable optically active acid or base, for example, tartaric acid.

The present invention also includes the use of all pharmaceutically acceptable isotopic variations of agents of the invention. An isotopic variation is defined as one in which at least one atom is replaced by an atom having the same atomic number, but an atomic mass different from the atomic mass usually found in nature.

Examples of isotopes suitable for inclusion in the agents of the invention include isotopes of hydrogen, such as ²H and ³H, carbon, such ¹³C and ¹⁴C, nitrogen, such as ¹⁵N, oxygen, such as ¹⁷O and ¹⁸O, phosphorus, such as ³²P, sulphur, such as ³⁵S, fluorine, such as ¹⁸F, and chlorine, such as ³⁶Cl.

Substitution of the agents of the invention with isotopes such as deuterium, i.e. ²H, may afford certain therapeutic advantages resulting from greater metabolic stability, for example, increased in vivo half-life or reduced dosage requirements, and hence may be preferred in some circumstances.

Certain isotopic variations of the agents of the invention, for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Isotopic variations of the agents of the invention can generally be prepared by conventional techniques known to those skilled in the art or by processes analogous to those described in prior art mentioned herein using appropriate isotopic variations of suitable reagents.

The agents of the invention may be freeze-dried, spray-dried, or evaporatively dried to provide a solid plug, powder, or film of crystalline or amorphous material. Microwave or radio frequency drying may be used for this purpose.

The agents of the invention may be administered alone or in combination with other drugs and will generally be administered as a formulation in association with one or more pharmaceutically acceptable excipients. The term “excipient” is used herein to describe any ingredient other than the compound of the invention. The choice of excipient will to a large extent depend on the particular mode of administration.

The agents of the invention may be administered orally. Oral administration may involve swallowing, so that the compound enters the gastrointestinal tract, or buccal or sublingual administration may be employed by which the compound enters the blood stream directly from the mouth. Formulations suitable for oral administration include solid formulations such as tablets, capsules containing particulates, liquids, or powders, lozenges (including liquid-filled), chews, multi- and nano-particulates, gels, films (including muco-adhesive), ovules, sprays and liquid formulations.

Liquid formulations include suspensions, solutions, syrups and elixirs. Such formulations may be employed as fillers in soft or hard capsules and typically comprise a carrier, for example, water, ethanol, propylene glycol, methylcellulose, or a suitable oil, and one or more emulsifying agents and/or suspending agents. Liquid formulations may also be prepared by the reconstitution of a solid, for example, from a sachet.

The agents of the invention may also be used in fast-dissolving, fast-disintegrating dosage forms such as those described in Expert Opinion in Therapeutic Patents, 11 (6), 981-986 by Liang and Chen (2001).

The composition of a typical tablet in accordance with the invention may comprise:

| Ingredient | % w/w |
|---|---|
| Agent of the invention | 10.00* |
| Microcrystalline cellulose | 64.12 |
| Lactose | 21.38 |
| Croscarmellose sodium | 3.00 |
| Magnesium stearate | 1.50 |

*Quantity adjusted in accordance with drug activity.

A typical tablet may be prepared using standard processes known to a formulation chemist, for example, by direct compression, granulation (dry, wet, or melt), melt congealing, or extrusion. The tablet formulation may comprise one or more layers and may be coated or uncoated.

Examples of excipients suitable for oral administration include carriers, for example, cellulose, calcium carbonate, dibasic calcium phosphate, mannitol and sodium citrate, granulation binders, for example, polyvinylpyrrolidine, hydroxypropylcellulose, hydroxypropylmethylcellulose and gelatin, disintegrants, for example, sodium starch glycolate and silicates, lubricating agents, for example, magnesium stearate and stearic acid, wetting agents, for example, sodium lauryl sulphate, preservatives, anti-oxidants, flavours and colourants.

Solid formulations for oral administration may be formulated to be immediate and/or modified release. Modified release formulations include delayed-, sustained-, pulsed-, controlled dual-, targeted and programmed release. Details of suitable modified release technologies such as high energy dispersions, osmotic and coated particles are to be found in Verma et al, Pharmaceutical Technology On-line, 25(2), 1-14 (2001). Other modified release formulations are described in U.S. Pat. No. 6,106,864.

The agents of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of agents of the invention used in the preparation of parenteral solutions may be increased by suitable processing, for example, preparation of an appropriate salt, the use of high energy spray-dried dispersions (see WO 01/47495) and/or by the use of appropriate formulation techniques, such as the use of solubility-enhancing agents.

Formulations for parenteral administration may be formulated to be immediate and/or modified release. Modified release formulations include delayed-, sustained-, pulsed-, controlled dual-, targeted and programmed release.

The agents of the invention may also be administered topically to the skin or mucosa, either dermally or transdermally. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin and propylene glycol. Penetration enhancers may be incorporated—see, for example, J Pharm Sci, 88 (10), 955-958 by Finnin and Morgan (October 1999).

Other means of topical administration include delivery by iontophoresis, electroporation, phonophoresis, sonophoresis and needle-free or microneedle injection.

Formulations for topical administration may be formulated to be immediate and/or modified release. Modified release formulations include delayed-, sustained-, pulsed-, controlled dual-, targeted and programmed release. Thus compounds of the invention may be formulated in a more solid form for administration as an implanted depot providing long-term release of the active compound.

The agents of the invention may also be administered intranasally or by inhalation, typically in the form of a dry powder (either alone, as a mixture, for example, in a dry blend with lactose, or as a mixed component particle, for example, mixed with phospholipids) from a dry powder inhaler or as an aerosol spray from a pressurised container, pump, spray, atomiser (preferably an atomiser using electrohydrodynamics to produce a fine mist), or nebuliser, with or without the use of a suitable propellant, such as dichlorofluoromethane.

The pressurised container, pump, spray, atomizer, or nebuliser contains a solution or suspension of the active compound comprising, for example, ethanol (optionally, aqueous ethanol) or a suitable alternative agent for dispersing, solubilising, or extending release of the active, the propellant(s) as solvent and an optional surfactant, such as sorbitan trioleate or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

A suitable solution formulation for use in an atomiser using electrohydrodynamics to produce a fine mist may contain from 1 μg to 10 mg of the compound of the invention per actuation and the actuation volume may vary from 1 μl to 100 μl. A typical formulation may comprise an agent of the invention, propylene glycol, sterile water, ethanol and sodium chloride. Alternative solvents which may be used instead of propylene glycol include glycerol and polyethylene glycol.

Capsules, blisters and cartridges (made, for example, from gelatin or HPMC) for use in an inhaler or insufflator may be formulated to contain a powder mix of the compound of the invention, a suitable powder base such as lactose or starch and a performance modifier such as l-leucine, mannitol, or magnesium stearate.

In the case of dry powder inhalers and aerosols, the dosage unit is determined by means of a valve which delivers a metered amount. Units in accordance with the invention are typically arranged to administer a metered dose or “puff” containing the agent of the invention. The overall daily dose will typically be administered in a single dose or, more usually, as divided doses throughout the day.

Formulations for inhaled/intranasal administration may be formulated to be immediate and/or modified release. Modified release formulations include delayed-, sustained-, pulsed-, controlled dual-, targeted and programmed release.

The agents of the invention may be administered rectally or vaginally, for example, in the form of a suppository, pessary, or enema. Cocoa butter is a traditional suppository base, but various alternatives may be used as appropriate.

Formulations for rectal/vaginal administration may be formulated to be immediate and/or modified release. Modified release formulations include delayed-, sustained-, pulsed-, controlled dual-, targeted and programmed release.

The agents of the invention may also be administered directly to the eye or ear, typically in the form of drops of a micronised suspension or solution in isotonic, pH-adjusted, sterile saline. Other formulations suitable for ocular and andial administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

Formulations for ocular/andial administration may be formulated to be immediate and/or modified release. Modified release formulations include delayed-, sustained-, pulsed-, controlled dual-, targeted, or programmed release.

The agents of the invention may be combined with soluble macromolecular entities such as cyclodextrin or polyethylene glycol-containing polymers to improve their solubility, dissolution rate, taste-masking, bioavailability and/or stability.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

For administration to human patients, the total daily dose of the agents of the invention is typically in the range 1 mg to 100 mg depending, of course, on the mode of administration. For example, oral administration may require a total daily dose of from 1 mg to 100 mg, while an intravenous dose may require a different amount. The total daily dose may be administered in single or divided doses.

These dosages are based on an average human subject having a weight of about 65 to 70 kg. The physician will readily be able to determine doses for subjects whose weight falls outside this range, such as infants and the elderly.

Example Formulations of Agents of the Invention are given below

Formulation 1: A tablet is prepared using the following ingredients:

| | weight/mg |
|---|---|
| Sildenafil citrate | 250 |
| Cellulose, microcrystalline | 400 |
| Silicon dioxide, fumed | 10 |
| Stearic acid | 5 |
| Total | 665 |

the components are blended and compressed to form tablets each weighing 665 mg

Formulation 2: An intravenous formulation may be prepared as follows:

| | | |
|---|---|---|
| Sildenafil citrate | 100 | mg |
| Isotonic saline | 1,000 | ml |

Formulation 3: A tablet is prepared using the following ingredients:

Sildenafil citrate (50 mg) is blended with cellulose (microcrystalline), silicon dioxide, stearic acid (fumed) and the mixture is compressed to form tablets.

1. A method of treating Type 1 Diabetes in a mammal suffering from Type 1 Diabetes comprising administering to the mammal a therapeutically effective amount of a selective PDE5 inhibitor, without substantial PDE2 inhibiting activity, or a pharmaceutically acceptable salt thereof, or a pharmaceutical composition containing either entity.

2. The method according to claim 1 wherein the PDE5 inhibitor is selected from sildenafil, tadalafil, vardenafil, DA-8159 and 5-[2-ethoxy-5-(4-ethylpiperazin-1-ylsulphonyl) pyridin-3-yl]-3-ethyl-2-[2-methoxyethyl]-2,6-dihydro-7H-pyrazolo[4,3-d]pyrimidin-7-one.

3. The method according to claim 1 or 2 further comprising one or more additional active agents selected from NO-agonist compounds or NO synthase substrates; potassium channel modulators; angiotensin receptor antagonists; antilipemic agents; antiplatelet or antithrombotic agents; acetylcholiesterase inhibitors; estrogen receptor modulators, agonists or antagonists; PDE inhibitors; NEP inhibitors; angiotensin-converting enzyme inhibitors or neutral endopeptidase, calcium-channel blockers; protein kinase C-β-inhibitors; activators of AMP-activated protein kinase; insulin; weight loss agents; dipeptidyl peptidase IV inhibitors; glucagons antagonists; inhibitors of PTP1B; reducers of PTP1B using antisense technology; glycogen synthase kinase-3 inhibitors; GLP-1 agonists; PPAR-gamma agonists; PPAR-alpha agonists; PPAR-alpha/PPAR-gamma agonists; sorbitol dehydrogenase inhibitors; reductase inhibitors; and soluble guanyl cyclase activators.

4. The method according to claim 3 wherein the active agent is selected from insulin, raloxifene, lasofoxifene, (−)-cis-6-phenyl-5-[4-(2-pyrrolidin-1-yl-ethoxy)-phenyl]-5,6,7,8-tetrahydronaphthalene-2-ol, atorvastatin, cerivastatin, fluvastatin, lovastatin, pravastatin, itavastatin, simvastatin and (+)-(3R,5S)-bis-(7-(4-(4-fluorophenyl)-6-isopropyl-2-(N-methyl-N-methanesulfonylamino)-pyrimidin-5-yl)-3,5-dihydroxy-6(E)-heptenoic acid.

5. The method according to claim 4 wherein the PDE5 inhibitor is sildenafil.

6. The method according to claim 4 wherein the agent is insulin,

7. The method according to claim 4 wherein the agent is raloxifene.

8. The method according to claim 4 wherein the agent is lasofoxifene.

9. The method according to claim 4 wherein the agent is (−)-cis-6-phenyl-5-[4-(2-pyrrolidin-1-yl-ethoxy)-phenyl]-5,6,7,8-tetrahydronaphthalene-2-ol.

10. The method according to claim 4 wherein the agent is atorvastatin.

11. The method according to claim 4 wherein the agent is cerivastatin.

12. The method according to claim 4 wherein the agent is fluvastatin.

13. The method according to claim 4 wherein the agent is lovastatin.

14. The method according to claim 4 wherein the agent is pravastatin.

15. The method according to claim 4 wherein the agent is itavastatin.

16. The method according to claim 4 wherein the agent is simvastatin.

17. The method according to claim 4 wherein the agent is (+)-(3R,5S)-bis-(7-(4-(4-fluorophenyl)-6-isopropyl-2-(N-methyl-N-methanesulfonylamino)-pyrimidin-5-yl)-3,5-dihydroxy-6(E)-heptenoic acid.

18. A pharmaceutical combination for the treatment of Type 1 Diabetes in an individual comprising an effective amount of a PDE5 inhibitor, without substantial PDE2 inhibiting activity, or a pharmaceutically acceptable salt thereof and one or more additional active agents selected from NO-agonist compounds or NO synthase substrates; potassium channel modulators; angiotensin receptor antagonists; antilipemic agents; antiplatelet or antithrombotic agents; acetylcholiesterase inhibitors; estrogen receptor modulators, agonists or antagonists; PDE inhibitors; NEP inhibitors; angiotensin-converting enzyme inhibitors or neutral endopeptidase; calcium-channel blockers; protein kinase C-β-inhibitors; activators of AMP-activated protein kinase; insulin; weight loss agents; dipeptidyl peptidase IV inhibitors; glucagons antagonists; inhibitors of PTP1 B; reducers of PTP1 B using antisense technology; glycogen synthase kinase-3 inhibitors; GLP-1 agonists; PPAR-gamma agonists; PPAR-alpha agonists; PPAR-alpha/PPAR-gamma agonists; sorbitol dehydrogenase inhibitors; reductase inhibitors; and soluble guanyl cyclase activators.

19. The pharmaceutical combination of claim 18 wherein the PDE5 inhibitor is selected from sildenafil, tadalafil, vardenafil, DA-8159 and 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one

20. The pharmaceutical combination according to claim 18 or 19 wherein the additional active ingredient is selected from antilipemic agents; estrogen receptor modulators, agonists and antagonists; and insulin.

21. The pharmaceutical combination according to claim 20 wherein the active ingredient is selected from insulin, raloxifene, lasofoxifene, (−)-cis-6-phenyl-5-[4-(2-pyrrolidin-1-yl-ethoxy)-phenyl]-5,6,7,8-tetrahydronaphthalene-2-ol, atorvastatin, cerivastatin, fluvastatin, lovastatin, pravastatin, itavastatin, simvastatin and (+)-(3R,5S)-bis-(7-(4-(4-fluorophenyl)-6-isopropyl-2-(N-methyl-N-methanesulfonylamino)-pyrimidin-5-yl)-3,5-dihydroxy-6(E)-heptenoic acid.

22. The combination according to claim 21 wherein the agent is insulin,

23. The combination according to claim 21 wherein the agent is raloxifene.

24. The combination according to claim 21 wherein the agent is lasofoxifene.

25. The combination according to claim 21 wherein the agent is (−)-cis-6-phenyl-5-[4-(2-pyrrolidin-1-yl-ethoxy)-phenyl]-5,6,7,8-tetrahydronaphthalene-2-ol.

26. The combination according to claim 21 wherein the agent is atorvastatin.

27. The combination according to claim 21 wherein the agent is cerivastatin.

28. The combination according to claim 21 wherein the agent is fluvastatin.

29. The combination according to claim 21 wherein the agent is lovastatin.

30. The combination according to claim 21 wherein the agent is pravastatin.

31. The combination according to claim 21 wherein the agent is itavastatin.

32. The combination according to claim 21 wherein the agent is simvastatin.

33. The combination according to claim 21 wherein the agent is (+)-(3R,5S)-bis-(7-(4-(4-fluorophenyl)-6-isopropyl-2-(N-methyl-N-methanesulfonylamino)-pyrimidin-5-yl)-3,5-dihydroxy-6(E)-heptenoic acid.

34. A kit for the treatment of Type 1 diabetes comprising a PDE5 inhibitor, without substantial PDE2 inhibiting activity, or a pharmaceutically acceptable salt thereof, in an effective amount, optionally one or more pharmaceutically acceptable carrier, excipient or diluent, and one or more of:
a. means for testing for Type 1 diabetes;
b. one or more additional active agents selected from NO-agonist compounds or NO synthase substrates; potassium channel modulators; angiotensin receptor antagonists; antilipemic agents; antiplatelet or antithrombotic agents; acetylcholiesterase inhibitors; estrogen receptor modulators, agonists or antagonists; PDE inhibitors; NEP inhibitors; angiotensin-converting enzyme inhibitors or neutral endopeptidase; calcium-channel blockers; protein kinase C-β-inhibitors; activators of AMP-activated protein kinase; insulin; weight loss agents; dipeptidyl peptidase IV inhibitors; glucagons antagonists; inhibitors of PTP1 B; reducers of PTP1 B using antisense technology; glycogen synthase kinase-3 inhibitors; GLP-1 agonists; PPAR-gamma agonists; PPAR-alpha agonists; PPAR-alpha/PPAR-gamma agonists; sorbitol dehydrogenase inhibitors; reductase inhibitors; and soluble guanyl cyclase activators; and/or
c. instructions for the treatment of Type 1 diabetes.

35. The kit of claim 34 wherein the PDE5 inhibitor is sildenafil, tadalafil, vardenafil, DA-8159 or 5-[2-ethoxy-5-(4-ethylpiperazin-1-ylsulphonyl)pyridin-3-yl]-3-ethyl-2-(2-methoxyethyl]-2,6-dihydro-7H-pyrazolo[4,3-d]pyrimidin-7-one.

36. The kit of claim 35 wherein the PDE5 inhibitor is sildenafil.

37. The kit of claims 34, 35 or 36 wherein the additional active agent is selected from: insulin, raloxifene, lasofoxifene, (−)-cis-6-phenyl-5-[4-(2-pyrrolidin-1-yl-ethoxy)-phenyl]-5,6,7,8-tetrahydronaphthalene-2-ol, atorvastatin, cerivastatin, fluvastatin, lovastatin, pravastatin, itavastatin, simvastatin and (+)-(3R,5S)-bis-(7-(4-(4-fluorophenyl)-6-isopropyl-2-(N-methyl-N-methanesulfonylamino)-pyrimidin-5-yl)-3,5-dihydroxy-6(E)-heptenoic acid.

38. The kit according to claim 37 wherein the agent is insulin,

39. The kit according to claim 37 wherein the agent is raloxifene.

40. The kit according to claim 37 wherein the agent is lasofoxifene.

41. The kit according to claim 37 wherein the agent is (−)-cis-6-phenyl-5-[4-(2-pyrrolidin-1-yl-ethoxy)-phenyl]-5,6,7,8-tetrahydronaphthalene-2-ol.

42. The kit according to claim 37 wherein the agent is atorvastatin.

43. The kit according to claim 37 wherein the agent is cerivastatin.

44. The kit according to claim 37 wherein the agent is fluvastatin.

45. The kit according to claim 37 wherein the agent is lovastatin.

46. The kit according to claim 37 wherein the agent is pravastatin.

47. The kit according to claim 37 wherein the agent is itavastatin.

48. The kit according to claim 37 wherein the agent is simvastatin.

49. The kit according to claim 37 wherein the agent is (+)-(3R,5S)-bis-(7-(4-(4-fluorophenyl)-6-isopropyl-2-(N-methyl-N-methanesulfonylamino)-pyrimidin-5-yl)-3,5-dihydroxy-6(E)-heptenoic acid.
